(19) United States
(12) Patent Application Publication
(10) Pub. No.: US 2010/0234836 A1
(43) Pub. Date: Sep. 16, 2010

(54) LED Based Light Surgery Apparatus

(75) Inventor: Sean Xiaolu Wang, Wilmington, DE (US)

Correspondence Address: BWT PROPERTY, INC., 19 SHEA WAY, SUITE 301, NEWARK, DE, 19713, US

(73) Assignee: BWT PROPERTY, INC., Newark, DE (US)

(21) Appl. No.: 12/720,744

(22) Filed: Mar. 10, 2010

Related U.S. Application Data
(60) Provisional application No. 61/158,843, filed on Mar. 10, 2009.

(51) Int. Cl. A61B 18/18

(52) U.S. Cl. 606/16; 606/13; 606/17

(57) ABSTRACT

A surgical apparatus for ablation, incision, and/or coagulation of biological tissue, the surgical apparatus comprising: at least one high intensity light emitting diode (LED) light source for producing a light beam with high power density; and an optical system for delivering said light beam to be absorbed by the biological tissue. The power density of the light beam is above a predetermined threshold level to increase a temperature of the biological tissue and cause a transformation for at least one constituent of the biological tissue for ablation, incision, and/or coagulation of the biological tissue.

### REFERENCE TO RELATED APPLICATION

This application claims an invention which was disclosed in Provisional Patent Application Number 61/158,843, filed Mar. 10, 2009, entitled “LED BASED LIGHT SURGERY APPARATUS”. The benefit under 35 USC §119(e) of the above mentioned United States Provisional Applications is hereby claimed, and the aforementioned application is hereby incorporated herein by reference.

### FIELD OF THE INVENTION

This invention generally relates to a light surgery apparatus, and more specifically to an LED based light surgery apparatus.

### BACKGROUND

A light surgery apparatus employs high intensity light to vaporize biological tissue for ablation or incision purposes. In comparison with conventional scalpel surgery, the light surgery causes less bleeding by coagulating the blood and cauterizing small blood vessels. In addition, the light surgery has a natural sterilization effect by vaporizing and killing bacteria, viruses and fungi. Up till today, lasers are the main kind of light sources used in light surgery, owing to their high intensity and good beam quality. Some examples are disclosed in U.S. Pat. Nos. 3,769,963, 6,757,310, and 7,452,355. However, laser based light surgery apparatus also suffer from large footprint, high power consumption, and high manufacturing and maintenance costs. There thus exists a need for an improved light surgery apparatus to overcome the above mentioned drawbacks of laser surgery apparatus.

### SUMMARY OF THE INVENTION

A surgical apparatus for ablation, incision, and/or coagulation of biological tissue, the surgical apparatus comprising: at least one high intensity light emitting diode (LED) light source for producing a light beam with high power density; and an optical system for delivering said light beam to be absorbed by the biological tissue. The power density of the light beam is above a predetermined threshold level to increase a temperature of the biological tissue and cause a transformation for at least one constituent of the biological tissue for ablation, incision, and/or coagulation of the biological tissue.

The LED based light surgery apparatus can be used as a replacement for laser surgery in incision, excision, vaporization, ablation, hemostasis, or coagulation of soft tissue in ear, nose, throat, and oral surgery (otolaryngology), dental procedures, arthroscopy, gastroenterology, general surgery, dermatology, plastic surgery, podiatry, urology, genitourinary, gynecology, neurosurgery (peripheral nervous system), pulmonary surgery, thoracic surgery, cosmetic surgery (removing tattoos, scars, stretch marks, sunspots, wrinkles, birthmarks, and hairs), spinal surgery, endovascular coagulation, removal of tumors, vascular surgery, eye surgery and refractive surgery, lipolysis and liposuction, etc.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 shows the schematic structure of one exemplary LED light surgery apparatus.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to an LED based light surgery apparatus. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

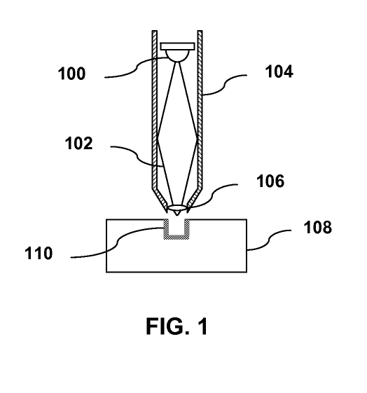

FIG. 1 shows one exemplary embodiment of the present invention. The light surgery apparatus comprise one or more high intensity light emitting diodes (LEDs) or superluminescent light emitting diodes (SLEDs) 100 as its light source. The LED light source 100 can be a surface-mounted or chip-on-board (COB) packaged LED, where the LED chips are directly surface mounted on a thermal conductive substrate for improved heat dissipation. The COB package allows larger light emitting surface and higher drive current for the LED chip to increase its output power. It also leads to long lifetime as well as wavelength and intensity stability. One advantage of LED light sources over lasers is that they are available in wider wavelength ranges, making it possible to match the LED output wavelength closely with the absorption band of the subject biological tissue. As a result, a lower power density can be used with LED light sources, which can help reduce unnecessary damages to the surrounding biological tissue. With the recent development of high intensity LED technology, the output power density of LEDs or SLEDs has improved significantly to meet the requirements of surgical applications. The LED light source 100 may operate in a continuous wave (CW) mode or in a pulsed mode. Operating the LED light source in a pulsed mode can further increase its peak output power density.

Referring back to FIG. 1, the LED light source 100 is enclosed in a tube 104, which has a reflective internal surface. The light beam 102 emitted by the LED light source 100 is reflected by the internal surface of the tube 104 to be delivered to an optical lens 106 positioned at the output end of the tube 104. The LED light is then focused by the optical lens 106 onto the surface of the subject biological tissue 108. The light beam can interact with the biological tissue through photochemical, photothermal, photoablation, or photodisruption mechanisms. Among them, photothermal interaction is the main mechanism for ablation, incision, or coagulation of the biological tissue, where the absorbed light energy is converted into heat and causes the temperature of the tissue to increase above certain threshold. The rise of tissue temperature causes transformation for certain constituent of the biological tissue, such as coagulation of blood, vaporization of water, denaturalization and carbonization of protein, etc. The water vaporization process induces pressure in the tissue and breaks the tissue into fragments which eventually vaporize along with the water. With even higher light intensity, the protein content of the tissue can be carbonized and vaporized as well. In FIG. 1, a section of the subject biological tissue 108 is ablated by the LED light with the periphery tissue 110 coagulated and cauterized to prevent any possible bleeding. Beyond the optical lens 106, other types of optical components (e.g. optical diffusers, focusing mirrors, non-imaging lenses, optical filters, polarizers) can be employed to enhance/modify the optical properties (brightness, divergence angle, beam size, intensity profile, wavelength, spectral bandwidth, coherence length, polarization, etc.) of the LED light such that an improved ablation, incision, or coagulation effect is produced.

In a slight variation of the present embodiment, the LED light can be first coupled into an optical waveguide and then delivered by the optical waveguide onto the subject biological tissue. The optical waveguide can be a glass/plastic optical fiber, a hollow waveguide, an articulated arm, or a liquid light guide. The optical waveguide may be embedded in a small cannula or catheter and inserted into the biological tissue to treat the inner layer of the tissue. One example of application is liposuction, where the LED light is used to melt, denaturalize, and destroy excessive lipid cells in the body. Another example is vein surgery, where the optical waveguide is inserted into the vein of the patient for treating internal structures of the vein.

In yet another variation of the present embodiment, the LED light source may comprise multiple LEDs with different output wavelengths. Each of the LED has an output wavelength matching with the absorption band of a specific constituent of the biological tissue. For example, LEDs with output wavelength at around 415 nm can be used for coagulation purposes as this wavelength coincides with the peak absorption band of hemoglobin, while near infrared (NIR) LEDs at 980 nm can be used for ablation purposes as the light in this wavelength is well absorbed by the water content of the tissue. It is also possible to use the LED light source in combination with laser light sources to obtain enhanced surgical results. For example, the light surgery apparatus may comprise a 980 nm laser for tissue ablation in addition to a 415 nm LED for performing coagulation.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. A surgical apparatus for ablation, incision, and/or coagulation of biological tissue, the surgical apparatus comprising:
at least one high intensity light emitting diode (LED) light source for producing a light beam with high power density; and
an optical system for delivering said light beam to be absorbed by the biological tissue;
wherein the power density of said light beam is above a predetermined threshold level to increase a temperature of the biological tissue and cause a transformation for at least one constituent of the biological tissue for ablation, incision, and/or coagulation of the biological tissue.

2. The surgical apparatus of claim 1, wherein said transformation comprises coagulation of blood content of the biological tissue.

3. The surgical apparatus of claim 1, wherein said transformation comprises vaporization of water content of the biological tissue.

4. The surgical apparatus of claim 1, wherein said transformation comprises denaturalization, carbonization, and vaporization of protein content of the biological tissue

5. The surgical apparatus of claim 1, wherein said transformation comprises melting, denaturalization, and vaporization of lipid content of the biological tissue.

6. The surgical apparatus of claim 1, wherein said optical system comprises optical components for modifying optical properties of said light beam.

7. The surgical apparatus of claim 6, wherein said optical properties comprises brightness, divergence angle, beam size, intensity profile, wavelength, spectral bandwidth, coherence length, and polarization properties.

8. The surgical apparatus of claim 1, wherein said optical system comprises a focusing lens.

9. The surgical apparatus of claim 1, wherein said optical system comprises an optical waveguide.

10. The surgical apparatus of claim 9, wherein said optical waveguide comprises an optical fiber, an articulated arm, or a liquid light guide.

11. The surgical apparatus of claim 1, wherein said LED light source comprises multiple LEDs with different output wavelengths.

12. The surgical apparatus of claim 1, wherein said LED light source operates in a continuous wave (CW) mode.

13. The surgical apparatus of claim 1, wherein said LED light source operates in a pulsed mode.

14. A method for ablation, incision, and/or coagulation of biological tissue, the method comprising the steps of:
providing at least one high intensity light emitting diode (LED) light source for producing a light beam with high power density; and
delivering said light beam to be absorbed by the biological tissue;
wherein the power density of said light beam is above a predetermined threshold level to increase a temperature of the biological tissue and cause a transformation for at least one constituent of the biological tissue for ablation, incision, and/or coagulation of the biological tissue.
